(19) United States
(12) Patent Application Publication
(10) Pub. No.: US 2008/0261199 A1
(43) Pub. Date: Oct. 23, 2008

(54) RAPID DETECTION PROCESSES AND RELATED COMPOSITIONS

(75) Inventors: Zhuying WANG, Dayton, NJ (US); Fangliang ZHANG, Fanwood, NJ (US)

Correspondence Address: ECKERT SEAMANS CHERIN & MELLOTT, 600 GRANT STREET, 44TH FLOOR, PITTSBURGH, PA, 15219, US

(73) Assignee: GENSCRIPT CORPORATION, Piscataway, NJ (US)

(21) Appl. No.: 11/678,989

(22) Filed: Feb. 26, 2007

Related U.S. Application Data
(60) Provisional application No. 60/794,999, filed on Apr. 26, 2006.

(51) Int. Cl. C12Q 1/70; G01N 33/53; C12Q 1/68; G01N 33/569

(52) U.S. Cl. 435/5; 435/7.1; 435/7.92; 435/6; 435/7.21

(57) ABSTRACT

An improved detection method is described for an antigen such as a chemical compound, a peptide, or a nucleic acid. The detection time for an antigen can be dramatically reduced relative to conventional technologies. The technology can particularly be used, for example, to modify and reduce the detection time significantly in traditional Western blot, Dot blot, ELISA and Immunohistology methods.

### CROSS REFERENCE TO RELATED APPLICATIONS

This application claims priority to U.S. Provisional Application Ser. No. 60/794,999, filed Apr. 26, 2006, entitled, “Rapid Detection Processes And Related Compositions,” the disclosure of which is incorporated herein by reference.

### TECHNICAL FIELD

The invention relates to a detection method for an antigen such as a chemical compound, or a peptide, or a protein, an RNA, a DNA, a cell, or a virus particle. In particular, the invention provides a method and composition useful for performing Western blots, Dot blots, ELISAs and Immunohistochemistry assays.

### BACKGROUND

Immunological methods have become important tools used to detect antigens including, for example, peptides, proteins, nucleic acids, biological cells, and virus particles. A wide variety of methods have been developed for the detection or quantitation of antigens. Among them, Western Blot, Dot Blot, ELISA and Immunohistology are the four most commonly used methods.

Western Blot, given to the technique by W. Neal Burnette (Analytical Biochemistry, 112:195-203, 1981), is a method for identifying and characterizing antigens. This technique is used to detect antigens by combining the resolution of antigens using gel electrophoresis with the specificity of immunodetection using a detection agent. Typically, a conventional Western blot (see, e.g., FIG. 1) is comprised of the following steps: (i) sodium dodecyl sulfate polyacrylamide gel electrophoresis (SDS-PAGE): antigens are resolved by relative mobility shift using electrophoresis; (ii) the resolved antigens are transferred from the SDS-PAGE gel from Step (i) onto a membrane; (iii) the membrane from Step (ii) is incubated with a blocking reagent for 1 hour to block non-specific binding sites in the membrane; (iv) the membrane is removed from the blocking solution and washed three times for 10 min each in PBS (phosphate buffered saline) or PBST (PBS containing 0.1% Tween-20); (v) the membrane from Step (iv) is incubated with a primary detection agent diluted in a solution for 1 hour. The primary detection agent binds the target antigen; (vi) the membrane from Step (v) is removed from the primary detection agent solution and washed three times for 10 min each in PBS or PBST to remove the non-specifically bound primary detection agent; (vii) the membrane from Step (vi) is incubated with a secondary detection agent diluted in a solution for 1 hour. The secondary detection agent binds the primary detection agent. The secondary detection agent can be, but is not limited to, a detection agent linked (coupled) to a reporter enzyme such as alkaline phosphatase (AP) or horseradish peroxidase (HRP), which can be detected visually through the conversion of a calorimetric substrate (chromagen) to a colored precipitate at the site of a detection agent binding; (viii) the membrane from Step (vii) is removed from the secondary detection agent solution and washed three times for 10 min each in PBS or PBST to remove the non-specifically bound secondary detection agent; (ix) a detection system such as luminescence or calorimetric system or other methods is used to detect the bound secondary detection agent. The duration of time, from Step (iii) to (ix) as described above, generally takes about 4.5 hours (seven steps).

In a typical conventional Western blot, the steps from Step (iii) to Step (ix) are performed on an orbital shaker or rocker. A typical conventional Western blot involves two incubation steps: one is the incubation between the membrane and the primary detection agent; another one is the incubation between the membrane and the secondary detection agent. Each incubation step usually takes about one hour. The incubation step is a two-phase reaction, and involves the binding reaction between the antigen on the membrane and the detection agent.

Dot blot is also an immunological technique for detecting, analyzing, and identifying antigens. The difference between Dot blot and Western Blot is that in Dot blot samples are spotted directly onto a membrane. After samples are spotted onto the membrane, they are probed and visualized in the same way as Western blot.

Typically, a conventional Dot blot (see, e.g., FIG. 3) is comprised of the following steps: (i) applying a sample to a membrane to allow the sample to bind to the membrane; (ii) the membrane from Step (i) is incubated with a blocking reagent for 1 hour to block non-specific binding sites in the membrane; (iii) the membrane from Step (ii) is removed from the blocking solution and washed three times for 10 min each in PBS or PBST; (iv) the membrane from Step (iii) is incubated with a primary detection agent diluted in a solution for 1 hour. The primary detection agent binds the target antigen; (v) the membrane from Step (iv) is removed from the primary detection agent solution and washed three times for 10 min each in PBS or PBST to remove the non-specifically bound primary detection agent; (vi) the membrane from Step (v) is incubated with a secondary detection agent diluted in a solution for 1 hour. The secondary detection agent binds the primary detection agent. The secondary detection agent can be, but is not limited to, a detection agent linked (coupled) to a reporter enzyme such as alkaline phosphatase (AP) or horseradish peroxidase (HRP), which can be detected visually through the conversion of a calorimetric substrate (chromagen) to a colored precipitate at the site of a detection agent binding; (vii) the membrane from Step (vi) is removed from the secondary detection agent solution and washed three times for 10 min each in PBS or PBST to remove the non-specifically bound secondary detection agent; and (viii) a detection system such as luminescence or calorimetric system is used to detect to the bound secondary detection agent.

In the Dot blot, the steps from Step (ii) to Step (vii) are performed on an orbital shaker or rocker. Dot blot involves two incubation steps: one is the incubation between the membrane and the primary detection agent; another one is the incubation between the membrane and the secondary detection agent. Each incubation step usually takes about one hour. The incubation step is a two-phase reaction, and involves the binding reaction between the antigen on the membrane and the detection agent.

ELISA (Enzyme-Linked ImmunoSorbant Assay or Antibody capture assay) is another commonly used method to detect and quantitate antigens or antibodies in a sample. There are two main variations on this method: the ELISA can be used to detect the presence of antigens that are recognized by a detection agent or it can be used to test for detection agents that recognize an antigen. There are many different types of ELISAs. Two of the most common types of ELISA are “Indirect ELISA” and “Sandwich ELISA.”

Typically, an Indirect ELISA (see, e.g., FIG. 5) is comprised of the following steps: (i) coat a solid phase with an antigen dissolved in a coating buffer; (ii) the solid phase from Step (i) is incubated with a blocking reagent for 1 hour to block non-specific binding sites on the solid phase; (iii) the solid phase from Step (ii) is washed three times with PBS or PBST for 1 to 10 minutes each; (iv) the solid phase from Step (iii) is incubated with a primary detection agent diluted in a solution for 1 hour; (v) the solid support from Step (iv) is washed three times for 1 min in PBS or PBST to remove the non-specifically bound primary detection agent; (vi) the solid support from step (v) is incubated with a secondary detection agent diluted in a solution for 1 hour. The secondary detection agent binds the primary detection agent. The secondary detection agent can be, but not limited to, a detection agent linked (coupled) to a reporter enzyme such as alkaline phosphatase (AP) or horseradish peroxidase (HRP), which can convert a colorless substrate to a colored product whose optical densities can be measured on an ELISA plate reader at target wavelengths; (vii) the solid support from Step (vi) is washed five times for 1 min each in PBS or PBST to remove the non-specifically bound secondary detection agent; and (viii) a detection system such as UV, Fluorescence, chemiluminescence or other methods is used to detect the bound secondary detection agent.

Indirect ELISA involves two incubation steps: one is the incubation between the solid support and the primary detection agent; another one is the incubation between the solid support and the secondary detection agent. Each incubation step usually takes about one hour. The incubation step is a two-phase reaction, and involves the binding reaction between the antigen on the solid support and the detection agent.

Sandwich ELISA (Enzyme-Linked ImmunoSorbant Assay, or Two-detection agent assays) is another commonly used method to detect and quantitate antigens in a sample. There are two main variations on this method: the ELISA can be used to detect the presence of antigens that are recognized by a detection agent or it can be used to test for a detection agent that recognizes an antigen.

Typically, a Sandwich ELISA (see, e.g., FIG. 7) is comprised of the following steps: (i) coat a solid phase with a primary detection agent dissolved in a coating buffer; (ii) the solid phase from Step (i) is incubated with a blocking reagent to block non-specific binding sites in the solid phase; (iii) the solid phase from Step (ii) is washed three times with PBS or PBST for 1 min each; (iv) the sample containing antigen is applied to the solid phase from Step (iv) to allow the antigen to bind the primary detection agent for 1 hour; (v) the solid phase from Step (iv) is washed three times with PBS or PBST for 1 min each to remove the non-specifically bound antigen; (vi) the solid phase from Step (v) is incubated with a secondary detection agent diluted in a solution for 1 hour; (vii) the solid phase from Step (vi) is washed three times for 1 min each with PBS or PBST to remove the non-specifically bound secondary detection agent; (viii) the solid phase from Step (vii) is incubated with a tertiary detection agent diluted in a solution for 1 hour; and (ix) a detection system such as UV, Fluorescence, chemiluminescence or other methods is used to detect bound tertiary antibody.

Sandwich ELISA involves three incubation steps: one is the incubation between the solid phase and the primary detection agent; the second one is the incubation between the solid phase and the secondary detection agent. another one is the incubation between the solid phase and the tertiary detection agent. Each incubation step usually takes about one hour. The incubation step is a two-phase reaction, and involves the binding reaction between the antigen on the solid phase and the detection agent.

Immunohistology is a technique to detect antigens directly in cells or tissues. When labeled detection agents are used to detect antigens in cells or tissues, several characteristics of an antigen can be readily determined. Most importantly, cell staining will demonstrate both the presence and subcellular localization of an antigen. Double-labeling techniques permit the simultaneous detection of two antigens, allowing comparisons of the relative distribution of different antigens. Many cell staining methods can also be used in conjunction with conventional histological stains and autoradiographic methods to compare the localization of the antigen with other markers. Cell staining can also be used in pathology studies for determining such variables as the type of infectious organism, the progenitor of a neoplastic cell, or the presence of an inflammatory response.

Typically, Immunohistology staining (see, e.g., FIG. 9) is comprised of the following steps: (i) Tissue samples (frozen sections, paraffin sections, resin sections, cell smears or cytopreps, etc.) are prepared; (ii) the sample from Step (i) is incubated with a blocking reagent for at least one hour (up to 4 or 5 hours) at room temperature (RT); (iii) the sample from Step (ii) is incubated in diluted primary detection agent for at least one hour (up to 4 or 5 hours) at room temperature (RT); (iv) the sample from Step (iii) is washed three times with PBS or PBST for 10 min each to remove the non-specifically bound primary detection agent; (v) the sample from step (iv) is incubated in diluted secondary detection agent at room temperature for at least one hour (up to three hours). The secondary detection agent binds the primary detection agent. The secondary detection agent can be, but not limited to, a detection agent linked (coupled) to a reporter fluorescent dye such as fluorosein, which can be detected visually under a fluorescent microscope; (vi) the sample from Step (v) is washed three times for 10 min each in PBS or PBST to remove the non-specifically bound secondary detection agent; and (vii) the sample from Step (vii) can be detected using a variety of methods such as microscope or fluorescence cell sorter (FACS).

In the Immunohistology method described above, the steps from Step (ii) to Step (v) can be performed on an orbital shaker or rocker. Immunohistology involves two incubation steps: one is the incubation between the sample and the primary detection agent; another one is the incubation between the sample and the secondary detection agent. Each step usually takes about one hour. The incubation step is a two-phase reaction, and involves the binding reaction between the antigen on the sample and the detection agent.

A conventional Western blot, as described above, takes about 4.5 hours, with at least 6 steps. In a conventional Western blot, each incubation step takes about 1 hour. A conventional Dot blot, as described above, takes about 4.5 hours, also having at least six steps. In a conventional Dot blot, each incubation step takes about 1 hour. A conventional Indirect ELISA, as described above, takes about 4.5 hours, and there are at least six steps. In a conventional Indirect ELISA, each incubation step takes about 1 hour. A conventional Sandwich ELISA, as described above, takes about 5.5 hours, and there are at least eight steps. In a conventional Sandwich ELISA, each incubation step takes about 1 hour. A conventional Immunohistology staining, as described above, takes at least 4.0 hours, and there are at least six steps. In a conventional Immunohistology staining, each incubation step (step 3 or step 5) takes about 1 hour. Since conventional Western blot, Dot blot, Indirect ELISA, Sandwich ELISA and Immunohistology analysis consume valuable time, there is a need for a simple and rapid process to address the problem.

Since the introduction by W. Neal Burnette (Analytical Biochemistry, 112:195-203, 1981) as a method for identifying and characterizing antigens, Western blot became the most commonly used method for protein detection and identification.

In 1971, Engvall and Perlmann (Immunochem., 8:871-874, 1971) coined the term “enzyme-linked immunosorbent assay” which is better known by the acronym, “ELISA”, to describe an enzyme-based immunoassay method which is very useful for measuring antigen concentrations.

A variation of ELISA, the Dot blot method, was reported by Hawkers R., Niday E. & Gordon J. (Anal. Biochemistry 119, 142-147, 1982) which can also be used for detecting and measuring antigen concentrations.

Although there have been substantive improvements in all these immuno-detection methods, including the quality of reagents, solid phase membranes and plastics, microplate readers, washers, and statistical software, the basic methodology has remained virtually unchanged.

The primary detection agent (for example, a primary antibody) can be labeled directly with reporter molecules such as HRP and used in Western blot, Dot blot, ELISA and Immunohistology, thus to remove the reporter molecule labeled secondary detection agent binding and washing steps and reduce the assay time (Histochemistry. 80(2):103-106, 1984). However, it is not practical or possible to label all the primary antibodies on the market. Further more, in some cases, the labeling of primary antibodies will result in lower affinity for the corresponding antigens.

### SUMMARY

An object of the invention is to provide an improved rapid Western blot method for the detection of antigens by integrating conventional Western blot step (iii) to (viii) (for example, into one step or two steps). Accordingly, the time can be reduced from 4.5 hours to about 2 hours or less (e.g., about one hour or less).

It is a further object of the invention to provide an improved rapid Western blot method by using vibration with rotation or shaking for the more efficient incubation between the membrane and the detection agent. Accordingly, the time for incubation can be reduced to less than 30 minutes (e.g., less than about 10 minutes).

It is a further object of the invention to provide a Dot blot method for the detection of antigens by integrating conventional dot blot step (ii) to (vii) into one or two steps. The time, accordingly, can be reduced from 4.5 hours to about 2 hours or less (e.g., about one hour or less).

It is a further object of the invention to provide an improved rapid Dot blot method by using vibration with rotation or shaking for the more efficient incubation between the membrane and detection agent. Accordingly, the time for incubation can be reduced to less than 30 minutes (e.g., less than about 10 minutes).

It is a further object of the invention to provide an Indirect ELISA method for the detection of antigens by integrating conventional Indirect ELISA step (ii) to (vii) into one or two steps. In this way, the time can be reduced from 4.5 hours to about 2 hours or less (e.g., about one hour or less).

It is a further object of the invention to provide an improved rapid Indirect ELISA method by using vibration with shaking for the more efficient incubation between the solid phase and a detection agent. Accordingly, the time for incubation can be reduced to less than 30 minutes (e.g., less than about 10 minutes).

It is a further object of the invention to provide a Sandwich ELISA method and technique for the detection of antigens by integrating conventional Sandwich ELISA step (ii) to (viii) into one step or two steps. The time can be reduced from to about 2 hours or less (e.g., about one hour or less).

It is a further object of the invention to provide an improved rapid Sandwich ELISA method by using vibration with shaking for the more efficient incubation between the solid phase and a detection agent. Accordingly, the time for incubation can be reduced to less than 30 minutes (e.g., less than about 10 minutes).

It is a further object of the invention to provide an Immunohistology analysis method and technique for the detection of antigens by integrating conventional Immunohistology step (ii) to (vi) into one step or two steps. The time can be reduced from to about 2 hours or less (e.g., about one hour or less).

It is a further object of the invention to provide an improved rapid Immunohistology analysis method by using vibration with shaking for the more efficient incubation between the sample and a detection agent. Accordingly, the time for incubation can be reduced to less than 30 minutes (e.g., less than about 10 minutes).

The invention provides a method comprising incubating a substrate comprising a target with a solution comprising at least a primary and a secondary detection agent; washing the substrate; and detecting the target by measuring the primary and/or secondary detection agent on the substrate. The solution can comprise Na₂HPO₄, NaH₂PO₄, NaCl, a blocking agent, and the primary and/or secondary detection agent. In one aspect, the solution comprises Na₂HPO₄ in the range of about 2 g to 8 g per liter; the amount of NaH₂PO₄ in the range of about 1 gram to 5 gram per liter; the amount of NaCl in the range of about 3 gram to 10 grams per liter; wherein the blocking agent comprises BSA and/or Tween-20; The amount of BSA is in the range of about 5 grams to 30 grams per liter; and the amount of Tween-20 is in the range of about 0.1 grams to 10 grams per liter; the primary and/or secondary detecting agent comprises a reporter-conjugated molecule and wherein the pH is in the range of about 6.0 to about 8.0. The substrate can be a membrane, a tissue, or a solid phase. In another aspect, the target is selected from the group consisting of a polypeptide, a chemical, an RNA, a DNA, a cell, or a virus particle. In yet another aspect, the incubating comprises incubation on a vibration device and/or a shaking or rotational device. Targets can include an HIV antigen or an antigen associated with Lyme disease. The solution may comprise a tertiary detection agent.

The invention also includes a method for the detection of an antigen or a detection agent, comprising (a) incubating a substrate comprising a target in a solution containing both a primary detection agent and a secondary detection agent; (b) washing the substrate; and (c) detecting the substrate from Step (b) with a detection system to observe signals, wherein the incubation and washing can be conducted on a regular shaker or a vibrator with rotation or shaking.

Either of the foregoing methods can further comprise additional steps. Furthermore, either of the foregoing methods can comprise first blocking the substrate. Furthermore, the substrate comprising the target of any of the foregoing steps can be first incubated with a blocking agent prior to the incubating with the solution.

The invention also provides a solution comprising about 2 grams to 8 grams per liter of Na₂HPO₄, about 1 gram to 5 grams per liter of NaH₂PO₄, about 3 grams to 10 grams per liter of NaCl, about 5 grams to 30 grams per liter of BSA and/or about 0.1 grams to 10 grams per liter of Tween-20 and a pH of about 6 to 8. In one aspect, the solution comprises about 0.1 milligrams to 10 milligrams per liter of HRP-labeled secondary antibody.

The invention also provides a kit comprising a solution of the invention and one or more components for performing a Western Blot, a dot blot, an ELISA, or an immunohistochemistry technique.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 schematically illustrates procedures of a classical Western blot.

FIG. 2 schematically illustrates procedures of a Western blot process of the invention.

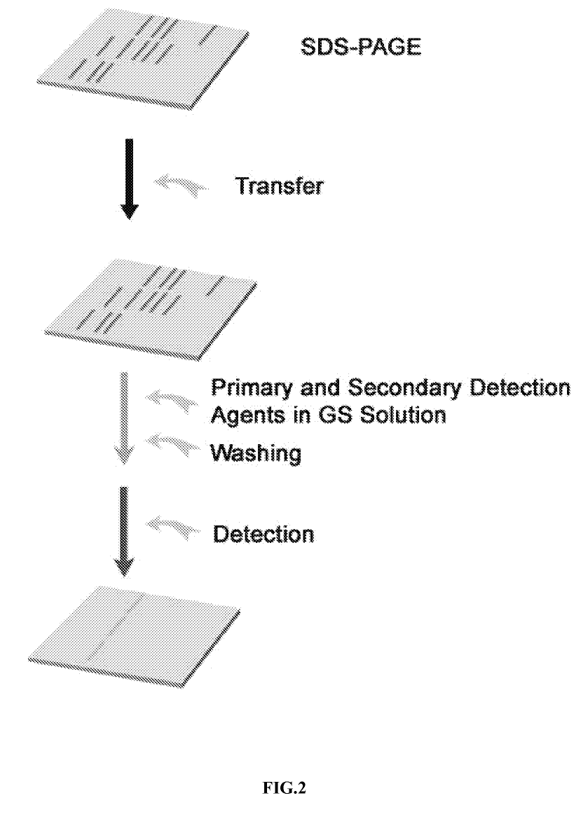

FIG. 3 schematically illustrates procedures of a classical Dot blot.

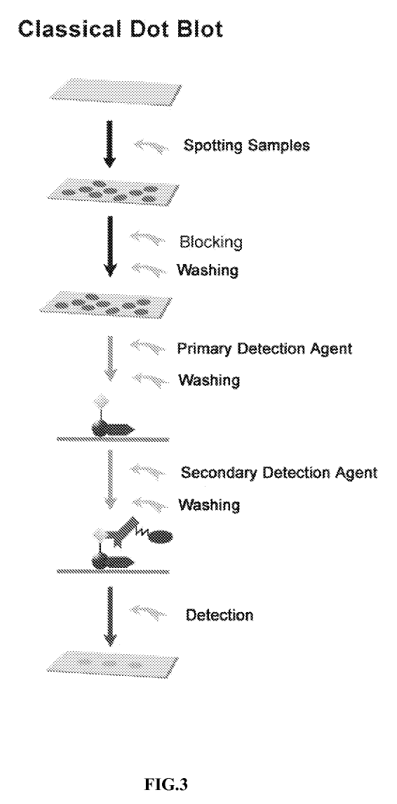

FIG. 4 schematically illustrates procedures of a Dot blot process of the invention.

FIG. 5 schematically illustrates procedures of a classical Indirect ELISA.

FIG. 6 schematically illustrates procedures of an Indirect ELISA process of the invention.

FIG. 7 schematically illustrates procedures of a classical Sandwich ELISA.

FIG. 8 schematically illustrates procedures of a Sandwich ELISA process of the invention.

FIG. 9 schematically illustrates procedures of a classical Immunohistology analysis.

FIG. 10 schematically illustrates procedures of Immunohistology analysis of the invention.

FIG. 11 shows an Example Western blot of the invention. Western blot for the detection of GST protein from E. coli cell lysate. 10 μl of original, 30 times diluted, 90 times diluted, 270 times diluted, 810 times diluted and 2500 times diluted E. coli cell lysate were loaded in Lane 1, Lane 2, Lane 3, Lane 4, Lane 5, Lane 6 and Lane 7, respectively. The blot was developed with BioFX TMB system (TMBM-0100-01).

FIG. 12 shows an Example Dot blot of the invention. Dot Blot for the detection of IL-8 protein. 1 μl of 100 ng/μl, 34 ng/μl, 14 ng/μl, 4.5 ng/μl, 1.5 ng/μl and 0.5 ng/μl of IL-8 protein were spotted to Dot 1, 2, 3, 4, 5 and 6, respectively. The blot was developed with BioFX TMB system (TMBM-0100-01).

FIG. 13 shows an example Indirect ELISA of the invention. Indirect ELISA for titration of antibody. The plate was coated with GST and GST polyclonal antibody was diluted as described in the table and tested in an Indirect ELISA assay.

FIG. 14 shows an example Sandwich ELISA of the invention. One-step Sandwich ELISA for the detection of GST protein. 0.01 ng, 0.1 ng, 1 ng and 10 ng of GST protein were added to plate wells precoated with rabbit anti-GST antibody.

### DETAILED DESCRIPTION

Unless defined otherwise, all technical and scientific terms used herein have the same meaning as is commonly understood by one of skill in the art to which this invention belongs. All publications and patents referred to herein are incorporated by reference.

The invention provides methods and compositions useful for performing antigen or antibody detection or diagnostics using Western blots, Dot blots, ELISA and Immunohistology. In one aspect, the invention provides a significant improvement of the conventional Western Blot, Dot Blot, ELISA and Immunohistology techniques. The invention provides a method whereby several steps in conventional Western Blot, Dot Blot, ELISA and Immunohistology are combined into one step or two steps. The methods of the invention greatly reduce the time for detection assays as well as associated costs.

Furthermore, the invention also provides a vibration technique and system combined with either rotation or shaking method to reduce the incubation time between an antigen on a solid phase or a membrane and a detection agent. This vibration combined with rotation or shaking can be applied to Western Blot, Dot blot, ELISA and Immunohistology.

As used herein “antigen” and an “antibody” are to be taken in their broadest context. An “antigen” can be any molecule, cell, virus, or particle. For example, an antigen includes, but is not limited to, a chemical molecule, a peptide molecule, a protein molecule, a RNA molecule, a DNA molecule, a traditional antibody, e.g., two heavy chains and two light chains, a recombinant antibody or fragment, a bacterial cell, a virus particle, a cell, a particle, and a product comprising crosslinking any two or more of the above. A target comprises an antigen and/or antibody to be detected.

An antigen can be a pure form, or it can also exist in a mixture. An antigen can be in a modified form (e.g., modified by a chemicals) or be an unmodified form.

Reference herein to an “antibody” is to be taken in their broadest context. “An antibody” is a polypeptide that binds to “an antigen”. An antibody includes, but is not limited to, a traditional antibody, a fragment of a traditional antibody containing an antigen binding site, a recombinant antibody containing an antigen binding site, a protein which binds to an antigen, and a product comprising crosslinking any two or more of the above.

An antibody can be in a pure form, or it can also exist in a mixture. An antibody can be in a modified form (e.g., modified by a chemical) or be an unmodified form.

The term “detection agent” refers to an agent that is used to detect an antigen or antibody. A detection agent could be either an “antigen” or an “antibody”. A detection agent could also be either a labeled “antigen” or “antibody” or an unlabeled “antigen” or “antibody”. There are a variety of labeling methods including, but not limited to, isotope labeling, chemical modification, enzyme conjugation, fluorescent dye labeling, luminescence and the like. Therefore, a detection agent includes, but is not limited to, a chemical molecule, a peptide molecule, a protein molecule, a RNA molecule, a DNA molecule, a traditional antibody, a fragment of a traditional antibody containing an antigen binding site, a recombinant antibody containing an antigen binding site, a protein which binds to an antigen, a bacterial cell, a viral particle, a cell, a particle, and a product comprising crosslinking any two or more of the above.

A detection agent can be in a pure form, or it can also be an impure form (e.g., contained in a mixture with other compounds or materials). A detection agent can be in a modified form or be an unmodified form. According to the order of a “detection agent” used in a method, a “detection agent” can be referred as “a primary detection agent” or “a secondary detection agent” or “a tertiary detection agent” or “a fourth detection agent”, and the like.

The term “detection system” refers to a system which can be used to give a readout comprising information related to the quantity or quality of a protein or agent in a sample (e.g., a blot, cell and the like) on a substrate. The choice of a detection system depends on the choice of the detection agent used in a method of the invention. For example, a detection system includes, but is not limited to, X-ray film or other beta/gamma sensitive material if the detection agent is isotope-labeled; if the detection agent is an enzyme-labeled, a chemical reaction which can result in color or chemiluminescence signal that can be detected by, for example, a CCD camera, visual inspection or other device capable of sensing a signal can be used; and if the detection agent is fluorescence-labeled, a fluorescence microscope or a fluorescence cell sorter can be used.

A substrate refers to a phase that comprises (e.g., binds) a target to be detected. Examples of such substrates include, but are not limited to, membranes such as nitrocellulose, PVDF or nylon; an ELISA solid phase such as polyacrylamide, plastic, metal, glass and the like; tissue culture samples such as tissue or cells on a glass, plastic or other material; and tissues derived from a subject.

A blocking agent refers to a reagent that binds to non-specific binding sites on a substrate. Examples include Tween-20, BSA or combinations thereof. In one aspect, BSA can be substituted with, for example, casein and/or other agents known in the art

The invention provides a composition useful in Western blots, Dot blots, ELISAs and Immunohistology. The composition is referred to herein as a GS solution. A GS solution of the invention comprises Na₂HPO4, NaH₂PO4, NaCl, Tween-20, BSA and a detection agent (labeled with a proper reporter molecule), for example. Each of these elements may be substituted with similar elements known in the art that function in solution in substantially the same way. BSA, for example can be substituted with casein and/or other agents known in the art. In one aspect, the GS solution comprises about 2 grams to 8 grams per liter of Na₂HPO₄ (e.g., about 3-7 grams, about 4-6 grams), but is typically about 5 grams. In another aspect, the GS solution comprises about 1 gram to 5 grams per liter of NaH₂PO₄ (e.g., about 1.5-4 grams, about 2-3 grams), but is typically about 2 grams. In yet a further aspect, the amount of NaCl in a liter of GS solution comprises about 0.1 grams to about 10 grams (e.g., about 0.5-8.5 grams, about 1-8 grams, about 2-7 grams, about 3-6 grams, 4-5 grams), typically about 1 gram), typically about 1 gram. In yet a further aspect, the amount of Tween-20 in a liter of GS solution comprises about 0.1 grams to about 10 grams (e.g., about 0.5-8.5 grams, about 1-8 grams, about 2-7 grams, about 3-6 grams, 4-5 grams), typically about 1 gram. In yet a further aspect, the amount of the detection agent in a liter of GS solution comprises about 0.1 milligrams to about 10 milligrams (e.g., about 0.5-8.5 milligrams, about 1-8 milligrams, about 2-7 milligrams, about 3-6 milligrams, about 4-5 milligrams), typically about 1 milligram. In yet a further aspect, the amount of BSA in a liter of GS solution is in the range of 5 to 30 grams, typically about 10 grams. For GS solution, the pH can be in the range of about 6.0-about 8.0 (e.g. about 6.8 to 7.4), but is typically at about 7.0.

A GS solution of the invention can be bottled and used as typically performed in research and diagnostic laboratories. GS solution is made in sterile water or distilled water that is sterile filtered and/or autoclaved. The GS solution of the invention can be used with Western blot assays, Dot blot assays, Immunohistology assays and ELISA assays. The GS solution can be included in an article of manufacture or kit for use in Western or Dot blots, ELISAs, Immunohistology and the like.

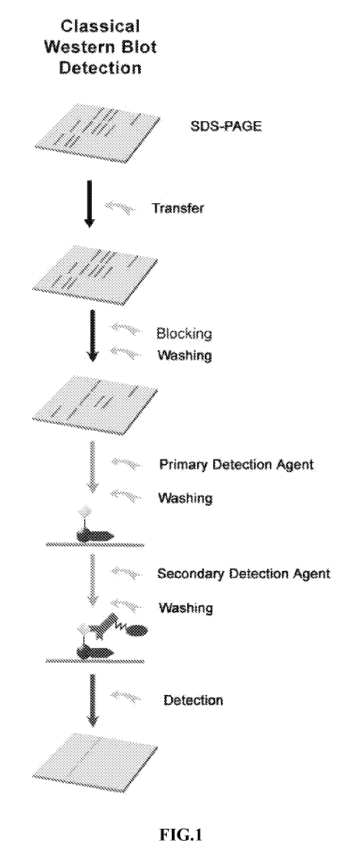

In one aspect, the invention provides a quick Western blot method similar (but with reduced steps) to a conventional Western blot. The method of the invention begins with sodium dodecyl sulfate polyacrylamide gel electrophoresis (SDS-PAGE). A protein sample comprising an antigen to be detected is generally first denatured by boiling (typically for about 5 minutes), and then the denatured proteins are resolved by SDS-PAGE as reported by U.K. Laemmli (Nature, 227: 680-685, 1970). Subsequent to electrophoresis, the resolved proteins in the gel are transferred to a membrane such as nitrocellulose, PVDF or nylon by means of the technique reported by W. Van Raamsdonk, et al. (J. Immunol. Methods, 17:337, 1977). Typically conventional Western blot is usually comprised of the steps enumerated, for example, in FIG. 1. For example, in a convention Western blot after the antigens are transferred onto the membrane, at least five steps are needed before the final wash and the detection step. Such steps comprise a blocking step, a washing step, a primary detection agent binding step, a washing step and a secondary detection agent binding step. Each of these steps is necessary in conventional Western blot to obtain acceptable results. The blocking step is to block remaining hydrophobic binding sites on the membrane to prevent non-specific protein binding of the detection agent used for detection of the target protein, thus to reduce background and/or to prevent false positive results. The subsequent wash step is to remove the blocking solution from the membrane. The primary detection agent and secondary detection agent are incubated with the membrane separately, and then washed away to avoid non-specific binding and to reduce the background. In the conventional Western blot, if any step is skipped during the procedure, there will be either very weak signal or too high background produced in the detection, which will lead to either false negative or false positive results.

In contrast, the invention provides a method whereby the blocking step and first washing step of conventional Western blot can be removed and the three steps of conventional Western blot (primary detection agent binding step, washing step and secondary detection agent binding step) are combined into one step. In the method of the invention a solution such as or comparable to GS solution is used. The method of the invention can greatly cut down the time required for Western blot analysis.

In conventional Western blot two incubation steps are utilized: one is the incubation between the membrane and the primary detection agent, another one is the incubation between the membrane and the secondary detection agent. The incubation step is a two-phase reaction, and involves the binding reaction between the antigen on the membrane and the detection agent. This incubation is usually performed on an orbital shaker or rocker, and is a rate-limiting step. In addition, each incubation step usually takes about one hour.

In a further aspect of the invention, an improved mixing method combining vibration and rotation, shaking or rocking can be used to reduce the time of incubation between the antigen on the membrane and the detection agent in the solution. For example, vibration combined with rotation, rocking or shaking facilitates the binding reaction between the antigen on the membrane and the detection agent, thus reducing the time required for incubation. Thus, the invention provides a method whereby the amount of time necessary to perform a blot can be reduced in at least two general areas by (i) combining incubation steps and (ii) reducing the incubation period by performing the incubation step with vibration.

The invention thus provides a method of protein identification using immunoblotting. In one aspect, the method includes resolving polypeptide antigens by sodium dodecyl sulfate polyacrylamide gel electrophoresis (SDS-PAGE) prior to blotting, followed by the transfer of the resolved polypeptides from the gel onto a membrane. The membrane is then processed by incubation with a solution such as GS solution comprising a primary detection agent (but will typically include a primary detection, secondary detection and blocking agent) for less than about 2 hour, then washed briefly with PBS or PBST. The incubation and washing can be performed on a regular shaker or a vibrational device comprising rotation, shaking or rocking. A detection system such as fluorescent, luminescent or calorimetric system or the like is used to detect a bound secondary detection agent.

In another embodiment of the invention, a blotting technique comprising resolving polypeptide antigens by sodium dodecyl sulfate polyacrylamide gel electrophoresis (SDS-PAGE), followed by the transfer of the resolved polypeptides from the gel onto a membrane. The membrane is the processed by incubation with a GS solution comprising a primary detection agent, a secondary detection agent and optionally a blocking agent for about 2 hour, typically less than 2 hours. It will be recognized that additional detection agents may be included as needed. The membrane is then washed briefly with PBS or PBST. The incubation and washing can be performed on a regular shaker or a device comprising a vibrational and a rotational or rocker action. Based upon the type of label used on the detection agent(s), a detection system such as fluorescent, luminescent or calorimetric system or the like is used to detect a bound secondary detection agent.

In the above-mentioned embodiments, SDS-PAGE is a method to separate antigens using polyacrylamide gel electrophoresis in the presence of sodium dodecyl sulfate (SDS) (Laemmli, 1970). Those skilled in the art will know how to prepare the gel and samples, and run the gel. Those skilled in the art will also know there are different methods of SDS-PAGE. These include but are not limited to: one-dimensional SDS-PAGE, two-dimensional SDS-PAGE, discontinuous gel electrophoresis, and continuous gel electrophoresis. Those skilled in the art will know how to load samples on SDS-PAGE gels and prepare proper buffers for running the SDS-PAGE gels.

In the above-mentioned embodiments, those skilled in the art will know how to transfer antigens from a polyacrylamide gel to a membrane. During the transferring process, the antigens are electrophoretically transferred in a tank or a semidry transfer apparatus to a nitrocellulose, PVDF, or nylon membrane, a process that can be monitored by reversible staining or by Ponceau S staining. The transferred proteins are bound to the surface of the membrane, providing access for reaction with immunodetection agents. Those skilled in the art will also know how to prepare transfer buffer for using in the transfer step.

The invention also provides a Dot blot method. Dot blots are also an immunological technique for detecting, analyzing, and identifying antigens (Hawkes, R. 1986, Methods Enzymol. 121: 484-491). The difference between a Dot blot and Western Blot is that in Dot blot protein samples are spotted directly onto a membrane and are not resolved by electrophoresis, although they may be purified to some extent prior to spotting. After samples are spotted onto the membrane, they are probed and visualized in the same way as a Western Blot.

In conventional Dot blot, after the antigens are spotted onto the membrane, at least five steps are needed before the final wash and detection step. These steps comprise blocking step, washing step, primary detection agent binding step, washing step and secondary detection agent binding step. Each of these steps is necessary in conventional Dot blot to obtain acceptable results. The blocking step is to block remaining hydrophobic binding sites on the membrane to prevent non-specific protein binding of the detection agent used for the detection of the target protein, thus to reduce background and/or to prevent false positive results. The subsequent wash step is to remove the blocking solution from the membrane. The primary detection agent and secondary detection agent are incubated with the membrane separately, and then washed away to avoid non-specific binding and to reduce the background.

In a conventional Dot blot, if any step is skipped during the procedure there will be either very weak signal or too high background produced in the detection, which will lead to either false negative or false positive results.

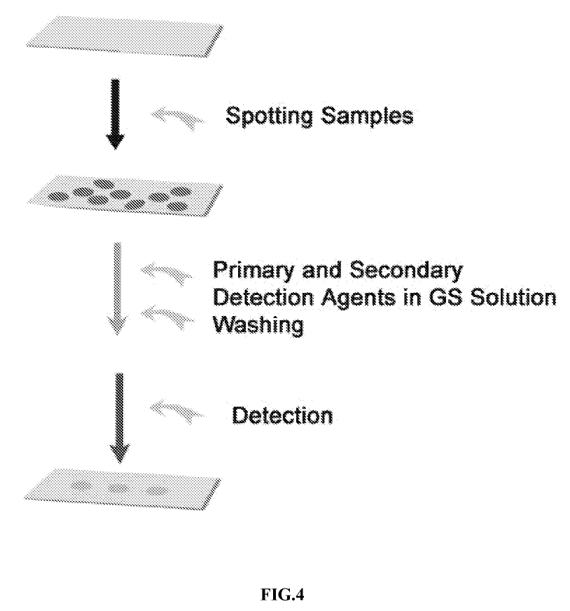

The invention provides a method for performing a Dot blot that comprises a reduced number of steps (see, e.g., FIG. 4). In the method of the invention, the blocking step and first washing step of a conventional dot blot can be removed, and the three steps of conventional Dot blot (primary detection agent binding step, washing step and secondary detection agent binding step) are combined into one step. In the method of the invention a formulated solution such as GS solution makes the reduced steps possible. The method of the invention will greatly cut down the time required for Dot blot analysis.

In another aspect, the invention provides a method of processing a protein detection system of the invention. As briefly mentioned above, the processing method includes a mixing method combining vibration and shaking of the membrane and detecting agents. The method reduces the incubation time between the antigen on the membrane and the detecting agents in the solution. In conventional Dot blot and Western Blots described above, a Dot blot comprises two incubation steps (i.e., one is the incubation between the membrane and the primary detection agent; another is the incubation between the membrane and the secondary detection agent). The incubation step is a two-phase reaction, and involves the binding reaction between the antigen on the membrane and the detection agent in the solution. This incubation step is usually performed on a shaker, and it is a rate-limiting step, and each incubation usually takes about one hour. In the invention, it was found that vibration combined with rotation could greatly facilitate the two-phase binding reaction between the antigen on the membrane and the detection agent in the solution, thus cut down the time required for incubation.

Thus, one embodiment of a Dot blot process of the invention comprise (i) applying a sample to a membrane to allow the sample to bind to the membrane (the sample may be a raw sample or a partially purified sample); (ii) incubating the membrane of (i) with a primary detection agent (e.g., a solution containing a primary detection agent for less than about 2 hour, then washed briefly). The solution can be, for example, a GS solution of the invention. The incubation and washing can be performed on a regular shaker or a vibrator with rotation or shaking. (iii) Detecting the presence of an antigen on the membrane with a detection system the measures, for example, fluorescence, luminescence, calorimetric or other signal to detect the bound secondary detection agent.

Another embodiment of a Dot blot technique of the invention comprises: (i) applying a sample to a membrane to allow the sample to bind to the membrane (the sample may be a raw sample of a partially purified or substantially purified sample); (ii) incubating the membrane of (i) with a primary and secondary detection agent in a solution containing both the primary detection agent and secondary detection agent (for about 2 hours or less), followed by washing. The solution can be, for example, a GS solution. The incubation and washing can be performed on a regular shaker or a vibrator with rotation or shaking. (iii) Detecting the presence of an agent on the membrane with a detection system the measures, for example, fluorescence, luminescence, calorimetric or other signal to detect to bound secondary detection agent.

In yet another embodiment of the invention, the Dot blot technique of the invention comprises (i) applying a sample to a membrane to allow the sample to bind to the membrane (the sample may be a raw sample of a partially purified or substantially purified sample); (ii) incubating the membrane of (i) with a primary, a secondary detection agent and a tertiary detection agent in a solution (for about 2 hours or less), followed by washing briefly. The solution can be, for example, GS solution. The incubation and washing can be performed on a regular shaker or a vibrator with rotation or shaking. (iii) Detecting the presence of an agent on the membrane with a detection system the measures, for example, fluorescence, luminescence, calorimetric or other signal to detect to bound secondary detection agent.

In the above-mentioned embodiments, those skilled in the art will know how to apply a sample to a membrane. The (e.g., a substrate the binds a target) can be, but is not limited to, a nitrocellulose, PVDF, or nylon membrane. Application of the sample to the membrane can be monitored by reversible staining or by Ponceau S staining. The antigens will bind to the surface of the membrane, providing access for reaction with immunodetection agents.

The invention also provides an ELISA (Enzyme-Linked ImmunoSorbant Assay) method, introduced by Engvall E and Perlman P (Immunochemistry. 8(9):871-4, 1971). ELISA is one of the most useful of the immunoassays. There are two main types of ELISA methods. One is indirect ELISA method, the other is Sandwich ELISA method.

Indirect ELISA (detection agent capture assay) is one of common ELISA methods. For detecting a detection agent (e.g. an antibody) that recognizes an antigen, the antigen is coated on the wells of microtiter plates with, and incubated with test solutions containing specific detection agents. Unbound detection agents are washed away. Then a second incubation with a solution containing a secondary detection agent (e.g. alkaline phosphatase conjugated to protein A, protein G, or antibodies against the detection agents of interest) is needed. After incubation, unbound labeled secondary detection agent is washed away. An incubation with a substrate of reporter enzyme may be also needed. A detection system such as UV, fluorescence, chemiluminescence or other methods is used to detect the bound secondary detection agent, which is proportional to the amount of the detection agent to be detected in the test solution.

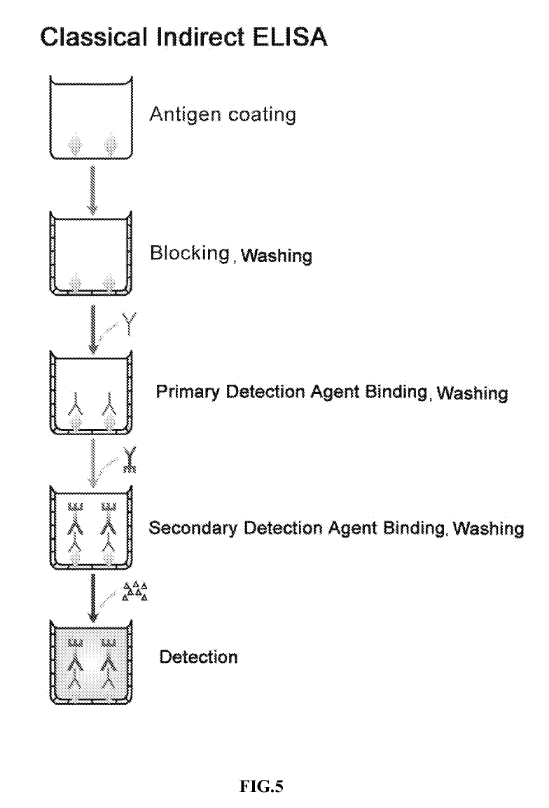

Typically, an Indirect ELISA is comprised of the steps enumerated in FIG. 5. In conventional Indirect ELISA, after the coating step and the wash step, five steps are needed before the final wash and the detection step. These steps comprise a blocking step, washing step, primary detection agent binding step, washing step and secondary detection agent binding step. Each of these steps is necessary in conventional Indirect ELISA to obtain acceptable results. The blocking step blocks remaining hydrophobic binding sites on the solid phase to prevent non-specific protein binding of the detection agent used for detection of the target protein, thereby reducing background and/or preventing false positive results. The subsequent wash step is to remove the blocking solution from the solid phase. The primary detection agent and secondary detection agent are incubated with the solid phase separately, and then washed away to avoid non-specific binding and to reduce the background.

In the conventional Indirect ELISA, if any step is skipped during the procedure, there will either be very weak signal or too high of a background produced in the detection, which will lead to either false negative or false positive results.

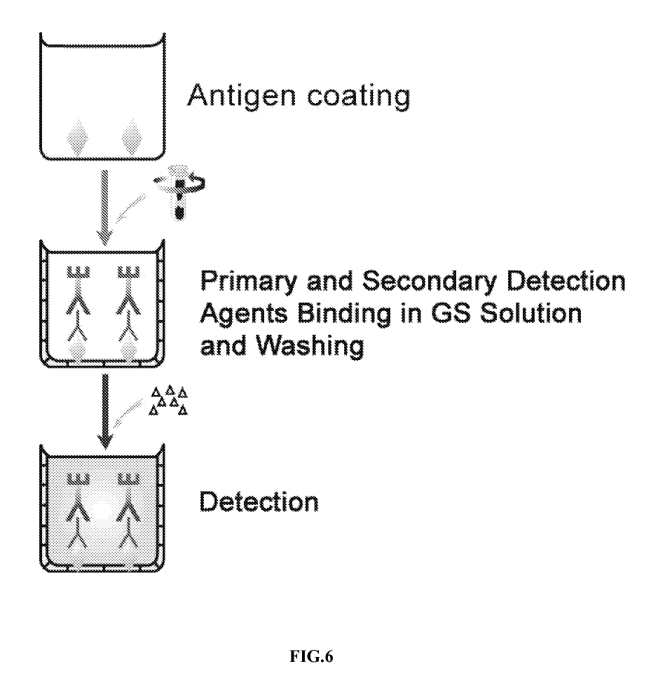

The invention provides an Enzyme-Linked ImmunoSorbant Assay that differs from conventional techniques in that the blocking step and first washing step of conventional Indirect ELISA can be removed, and the three steps of conventional Indirect ELISA (primary detection agent binding step, washing step and secondary detection agent binding step) are combined into one step (see, e.g., FIG. 6). In one aspect, the ELISA method of the invention uses a GS solution. The new method can greatly cut down the time required for Indirect ELISA analysis.

The ELISA method of the invention comprises (i) coating a solid phase with an antigen dissolved in a coating buffer; (ii) incubating the solid phase of (i) with a primary detection agent in a solution such as GS solution (for about 2 hours or less) followed by washing briefly in PBS or PBST. The incubation and washing can be performed on a regular shaker or a vibrator with shaking. (iii) detecting the presence of an agent on the solid phase with a detection system that measures, for example, UV, fluorescence, luminescence, calorimetric or other signal to detect the bound secondary detection agent.

In another embodiment of the invention, the ELISA of the invention comprises (i) coating a solid phase with an antigen dissolved in a coating buffer. (ii) Incubating the solid phase of (i) with a primary and secondary detection agent in a solution (e.g., a GS solution) for about 2 hours or less followed by a wash with, for example, PBS or PBST. The incubation and washing can be performed on a regular shaker or a vibrator with shaking. (iii) Detecting the presence of an agent on the solid phase with a detection system the measures, for example, UV, fluorescence, luminescence, calorimetric or other signal to detect to bound secondary detection agent.

In yet another embodiment of the invention, the ELISA method of the invention comprises (i) coating a solid phase with an antigen dissolved in a coating buffer; (ii) incubating the solid phase of (i) with a primary, secondary, and tertiary detection agent in a solution such as a GS solution (for about 2 hours or less). This can be followed by a brief wash. The incubation and washing can be performed on a regular shaker or a vibrator with shaking. (iii) Detecting the presence of an agent on the solid phase with a detection system the measures, for example, UV, fluorescence, luminescence, calorimetric or other signal to detect the bound secondary or tertiary detection agent.

In the above-mentioned embodiments, those skilled in the art will know how to coat a solid phase with an antigen or antigens. The antigens are usually dissolved in a binding buffer. There may be a variety of buffers for antigen binding. For example, a binding buffer can comprise 0.05 M carbonate buffer, pH 9.6 (other binding buffers are known in the art, e.g., PBS containing 0.1% Tween-20). The coating of the solid phase can be performed at different temperatures, e.g., from about 4° C. to room temperature to 37° C. The coating time can vary from 1 hour to overnight. In the above-mentioned embodiments, those skilled in the art will know how to wash the solid phase after coating. Different wash buffer can be used. A typical wash buffer comprises PBST (PBS solution containing 0.1% Tween-20) or water. The wash time can vary from a simple rinse or a 10 minute wash. The wash can also be performed multiple times.

Another aspect of this invention is to provide a quick Sandwich ELISA method. Sandwich ELISA (Enzyme-Linked ImmunoSorbant Assay, or two-detection agent assays) is commonly used to detect antigens in a sample. Sandwich ELISA may be the most useful of the immunosorbent assays for detecting antigen because of its sensitivity. To detect an antigen, the wells of microtiter plates are coated with specific (capture) detection agent (e.g., an antibody) followed by incubation with test solutions containing an antigen. Unbound antigen is washed away and a second antigen-specific detection agent conjugated to a detectable signal or signal-generating enzyme is added and incubated. Unbound conjugate is washed away and a substrate is added for signal development or detection is performed.

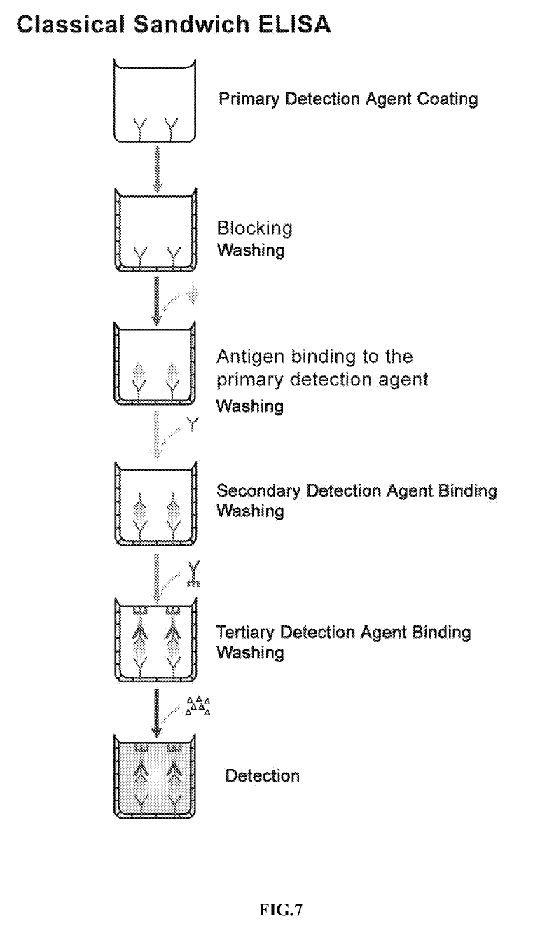

Typically, a Sandwich ELISA is comprised of the steps enumerated in FIG. 7. In conventional Sandwich ELISA, after the coating step, seven steps are needed before the final wash and the detection step. These steps comprise a blocking step, washing step, binding antigen to the primary detection agent, washing step, binding a second primary detection agent to the antigen, washing step, and binding secondary detection agent to the second primary detection agent. Each of these steps is necessary in conventional Sandwich ELISA to obtain acceptable results. The antigen and secondary detection agent are incubated with the solid phase separately, and then washed away to avoid non-specific binding and to reduce the background. In the conventional Sandwich ELISA, if any step is skipped during the procedure, there will be either very weak signal or too high background produced in the detection, which will lead to either false negative or false positive results.

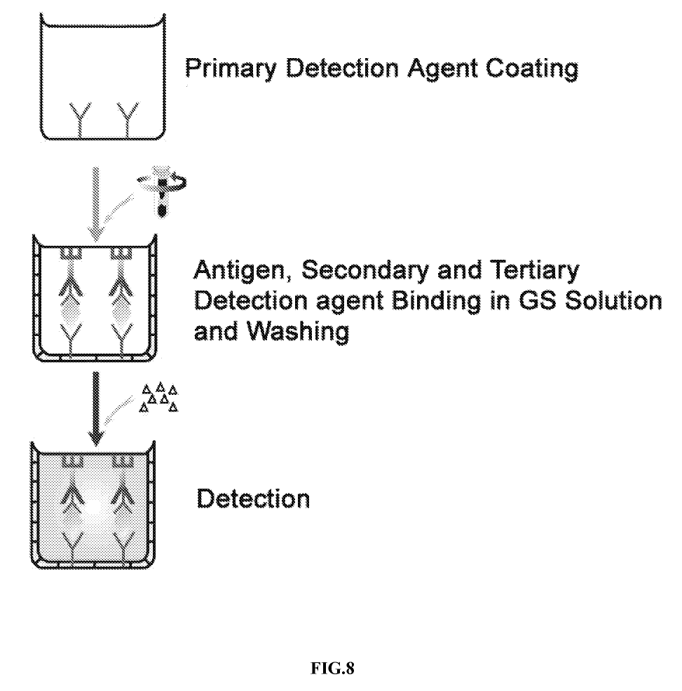

The invention provides an ELISA method whereby the number of steps and the length of time for performing the assay are reduced. In the method of the invention, the blocking step and first washing step of conventional Sandwich ELISA are removed, and the three steps of conventional Sandwich ELISA (primary detection agent binding step, washing step and secondary detection agent binding step, etc.) are combined into one step (see, e.g., FIG. 8). In this new method, a solution such as GS solution makes this possible. This new method will greatly cut down the time required for Sandwich ELISA assay.

Thus, in one embodiment of the invention, a Sandwich ELISA comprises (i) incubating a primary detection agent in coating buffer with the solid phase overnight; (ii) incubating the solid phase of (i) with antigen and the second primary detection agent in a solution such as GS solution (for about 2 hours or less) followed by brief washing with PBS or PBST. The incubation and washing can be performed on a regular shaker or a vibrator with shaking. (iii) Detecting the presence of an agent on the solid phase with a detection system the measures, for example, UV, fluorescence, luminescence, calorimetric or other signal to detect to bound secondary detection agent.

In another embodiment of the invention, the invention provides a Sandwich ELISA comprising a method of: (i) incubating a primary detection agent in coating buffer with a solid phase overnight; (ii) incubating the solid phase of (i) with an antigen, a primary detection agent, and a secondary detection agent in a solution such as GS (for about 2 hours or less), then washed briefly. The incubation and washing can be performed on a regular shaker or a vibrator with shaking. (iii) Detecting the presence of an agent on the solid phase with a detection system the measures, for example, UV, fluorescence, luminescence, calorimetric or other signal to detect to bound third detection agent.

In the above-mentioned embodiments, those skilled in the art will know how to coat a solid phase with an antigen or antigens. The antigens are usually dissolved in a binding buffer. There may be a variety of buffers for antigen binding (e.g., 0.05 M carbonate buffer, pH 9.6; or PBS). The coating of the solid phase can be performed at about 4° C. to about 37° C. The coating time can varies from about 1 hour to overnight. Washing after coating is necessary, a common wash buffer is PBST (PBS solution containing 0.1% Tween-20).

Immunohistology is a technique to detect antigens directly in cells or tissues. When labeled detection agents are used to detect antigens in cells or tissues, several characteristics of an antigen can be readily determined. Most importantly, cell staining will demonstrate both the presence and subcellular localization of an antigen. Double-labeling techniques permit the simultaneous detection of two antigens, allowing comparisons of the relative distribution of different antigens. Many cell staining methods can also be used in conjunction with conventional histological stains and autoradiographic methods to compare the localization of the antigen with other markers. Cell staining can also be used in pathology studies for determining such variables as the type of infectious organism, the progenitor of a neoplastic cell, or the presence of an inflammatory response.

The antigen is recognized by a primary detection agent that is added to the cells or tissues. Then a second incubation with a solution containing a secondary detection agent (e.g. alkaline phosphatase conjugated to protein A, protein G, or secondary antibodies against the primary detection agent) is used. After incubation, unbound labeled secondary detection agent is washed away. An incubation with a substrate of reporter enzyme may also be used. The immunologic reaction can be detected using a variety of methods such as microscopy (regular or automated) or fluorescence cell sorter.

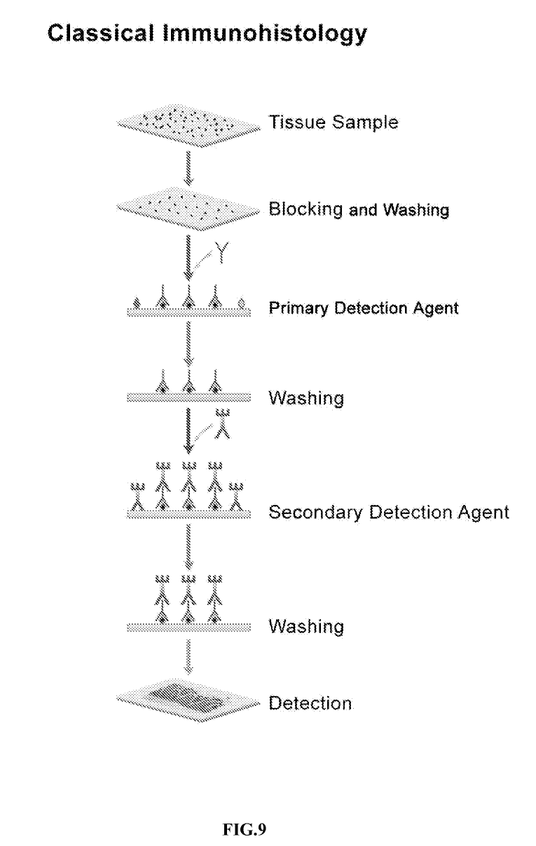

In conventional Immunohistology methods, as depicted in FIG. 9, five steps are needed before the final wash and the detection step. These steps comprise a blocking step, washing step, primary detection agent binding step, washing step and secondary detection agent binding step. Each of these steps is necessary in a conventional Immunohistology method to obtain acceptable results. The blocking step is to block non-specific binding sites on the tissue or cells to prevent non-specific protein binding of the detection agent used for detection of the target protein, thus to reduce background and/or to prevent false positive results. The primary detection agent and secondary detection agent are incubated with the tissue/cells separately, and then washed away to avoid non-specific binding and to reduce the background. In the conventional Immunohistology method, if any step is skipped during the procedure, there will be either very weak signal or too high background produced in the detection, which will lead to either false negative or false positive results.

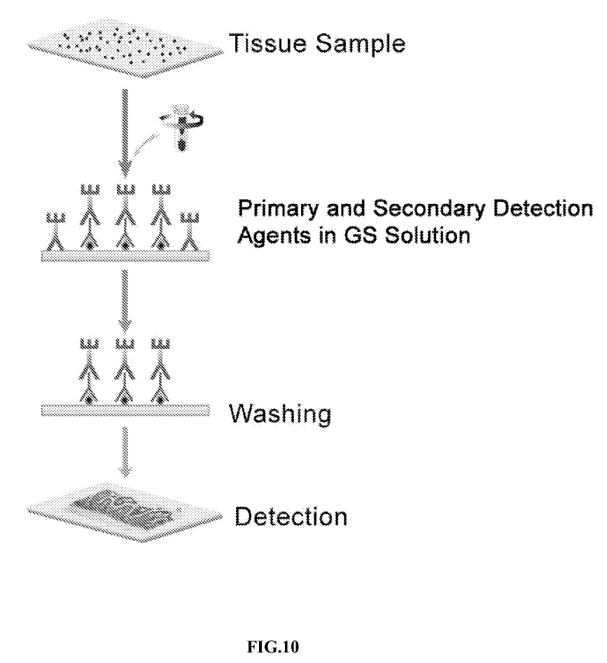

The invention provides a faster method of immunohistology. In the method of the invention the blocking step and washing step of conventional Immunohistology are removed, and the three steps of conventional Immunohistology (primary detection agent binding step, washing step and secondary detection agent binding step) are combined into one step (see, e.g., FIG. 10). In one aspect, the invention uses a GS solution as described herein.

The immunohistology method comprises preparing cells or tissue samples according to typical Immunohistology. This is followed by incubating with a primary detection agent in a solution such as GS solution (for about 2 hour or less), then washed briefly. The incubation can be performed without any movement or on a regular shaker or on a vibrator with shaking. A detection system such as fluorescence, chemiluminescence or other methods is used to detect a bound secondary detection agent.

In another embodiment of the invention, the immunohistology method of the invention comprises incubating a prepared tissue or cell sample with primary and secondary detection agent in a solution such as GS solution (for about 2 hour or less), then washed briefly. The incubation can be performed without any movement or on a regular shaker or on a vibrator with shaking. A detection system such as Fluorescence, chemiluminescence or other methods is used to detect the bound secondary detection agent.

In yet another embodiment of the invention, the immunohistology method comprises incubating a prepared cell or tissue sample with primary, secondary and tertiary detection agent in a solution such as GS solution for about 2 hour or less, then washed briefly. The incubation can be performed without any movement or on a regular shaker or on a vibrator with shaking. A detection system such as Fluorescence, chemiluminescence or other methods is used to detect the bound detection agent.

In the above-mentioned immunohistology embodiments, those skilled in the art will know how to prepare cells or tissues for immunohistology. The cells may or may not be fixed. The fixation can be performed, for example, in different solutions such as 2% paraformaldehye or 2% paraformaldehye/0.1% Triton X-100. The sample can be washed in PBS to remove fixation solution. Tissue samples may or may not need to be sectioned.

The methods and compositions of the invention can be used in a variety of applications. One common use of the method is for disease diagnostics. For example, the methods and compositions can be used to detect whether a patient has antibodies against HIV viral antigen(s). For example, HIV virus antigens can be resolved by gel electrophoresis and transferred onto a blotting membrane. A sample from a patient is processed and then mixed with a GS solution, and the mixture is then incubated with the membrane with resolved HIV viral antigens. If the sample from the subject has an antibody (primary antibody) to a viral antigen, the antibody will interact with the membrane at the location of the HIV viral antigens. A labeled secondary or tertiary detection agent (e.g., an anti-human antibody in the GS solution) then interacts with the bound primary antibody. The bound secondary detection agent or tertiary detection agent can be detected by a detection system. From the detection results, it can be concluded whether the patient has antibodies against the HIV virus.

Another example is to use methods and compositions of the invention to test whether a patient has antibodies against the pathogen that causes Lyme disease. Lyme virus antigens can be resolved by gel electrophoresis and transferred onto a membrane. A sample from a patient is processed then mixed with a GS solution, and the mixture is then incubated with the membrane. As above, if the subject's sample comprises antibodies against Lyme disease antigens, the antibodies (“primary”) will interact with the membrane at the antigen's location on the membrane. A secondary detection agent or tertiary detection agent then interacts with the bound primary antibody and can be detected by a detection system. From the detection results, it can be concluded whether the patient has antibodies against the pathogen causing Lyme disease.

In the above-mentioned embodiments, those skilled in the art will know how to select a detection agent for a specific antigen. A detection agent can be, but is not limited to, a chemical molecule, or a peptide molecule, or a protein molecule, or a RNA molecule, or a DNA molecule, or an antibody, or a fragment of an antibody, or a recombinant antibody, or a bacteria cell, or a virus particle, or a cell, or a particle. One of the most commonly used detection agents is an antibody. Antibodies can be derived from different species, and they include, but are not limited to, rabbit, mouse, rat, sheep, goat, and chicken antibodies. Commercially available antibodies to a wide variety of antigens are known in the art.

Another one of the more commonly used detection agents comprise protein A, G, L, A/G or other antibody-binding polypeptides. These polypeptides can bind to the conserved region in an antibody. Yet another one of the most used detection agents is avidin or strepavidin. Avidin or streptavidin can bind to biotinylated antibodies or polypeptides. The detection agent in the above-mentioned embodiments can be a labeled detection agent or an unlabelled detection agent.

In the above-mentioned embodiments, those skilled in the art will know that there are a variety of labeling methods for a detection agent. The labeling methods include, but are not limited to, an enzyme such as horseradish peroxidase (HRP) or alkaline phosphatase (AP) or beta-galactosidase or other enzymes. A detection agent can also be labeled with radioactive isotopes of iodine or other isotopes. A detection agent can also be labeled by a fluorochrome that can be detected by fluorescence microscope or fluorometer. A detection agent can also be labeled by a lumichrome which can be detected by luminescence methods. Alternatively, a detection agent can also be labeled by biotin, which can bind to avidin or streptavidin.

In the above-mentioned embodiments, those skilled in the art will be aware of different detection systems used in Western blots, ELISAs, Dot blots, and Immunohistology that can be applied to the methods of the invention described herein. These detection systems include, but are not limited to, detection system using X-ray films for radioactive isotope labeling or chemiluminescent reactions, detection system using chromogenic reactions of reporter enzymes such as HRP (Horseradish peroxidase) and AP (alkaline phosphatase). The reporter enzymes can use different substrates for either luminescent detection or chromogenic detection. For example, HRP can use 4 CN (4-chloro-1-napthol), DAB/NiCl₂ (3,3′-diaminobenzidine/NiCl₂), TMB as substrates for chromogenic detection. It can also use luminol/H₂O₂/p-iodophenol for chemiluminescent detection. For AP, BCIP/NBT (5-bromo-4-chloro-3-indolyl phosphate/nitroblue tetrazolium) can be used for luminescent detection. For chromogenic detection, the color will show up on the membrane and can be directly visualized. For chemiluminescent detection, the membrane needs to be exposed to a film or some other means for photon capture to visualize the results, or the membrane can be detected using CCD camera.

In the above-mentioned embodiments, those skilled in the art will know that other solutions can also be used in addition to GS solution provided in this invention. The purpose of GS solution is to improve the signal to noise ratio in immunodetection assays, which allows combining the primary detection agent binding step and secondary detection agent binding step into a single step. Alternatively, the primary, secondary, and tertiary detection agent binding step can all be combined into one step. It can be envisioned that other solutions which serve similar functions such as GS solutions can be developed.

In the above-mentioned embodiments, those skilled in the art will be aware of modifications to further improve the signal to noise ratio. These modifications include, but are not are limited to, adding one or multiple steps to the above embodiment. One of the examples is adding a blocking step.

Examples of blocking solutions and agents useful in the invention include, but are not limited to, tris-buffered saline containing 0.1% (v/v) Tween-20 (TTBS), or Tris-buffered saline containing 10% (w/v) non-fat milk, or TTBS containing 0.2% casein. The blocking solution may also contain one or more of reagents such as Triton X-100, or BSA, or fish gelatin, or casein, or other chemical reagents. The pH can be in the range of 2 to 14, typically from 7 to 8.

The invention also provides a quick and efficient blocking solution useful for performing a Western blot, an ELISA, a Dot blot, or an Immunohistochemical assays. The blocking solution may contain one or more of reagents such as Tween-20, or BSA, or fish gelatin, or casein, or other chemical reagents. The pH of the working solution can be in the range of 9 to 14, typically from 12.5 to 13.5.

In the above-mentioned embodiments, those skilled in the art will know that there are a variety of vibration and shaking (e.g., rotation) methods. The frequency of vibration can varies from 0 to 20,000 Hz, (e.g., from 0 to 2000 Hz, from 0 to 200 Hz). The speed of rotation or shaking can vary from 0 to 1400 rpm (rotation per minutes), typically from 0 to 140 rpm, or from 0 to 14 rpm.

The invention also provides kits comprising one or more components useful for performing a Western blot, an ELISA, a Dot blot, or an Immunohistochemical assays and instructions for carrying out a method of the invention. For example, such instructions can include methods for preparing a GS solution of the invention. In another aspect, the kit may be compartmentalized to receive a GS buffer and one or more components for performing a Western blot, an ELISA, a Dot blot or an Immunohistochemical assays. In yet another embodiment, the kit can comprise a device for incubation that delivers a vibration and shaking to a sample.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific examples of the methods of the invention are further illustrative of the nature of the invention, it needs to be understood that the invention is not limited thereto.

### EXAMPLES

### Example I

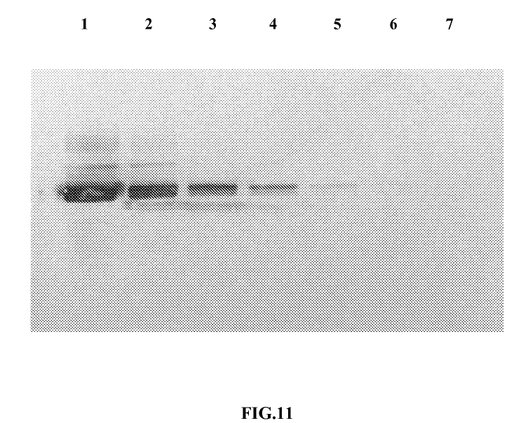

Western blot for the detection of GST protein from E. coli cell lysate. An E. coli cell culture expressing GST protein was harvested and lysed with SDS gel loading buffer. 10 μl of original, 30 times diluted, 90 times diluted, 270 times diluted, 810 times diluted and 2500 times diluted E. coli cell lysate were loaded onto a mini SDS-PAGE gel in Lane 1, Lane 2, Lane 3, Lane 4, Lane 5, Lane 6 and Lane 7, respectively. After electrophoresis, the proteins were transferred to nitrocellulose membrane. The membrane was then incubated in GS solution containing 1.5 μg/ml of Rabbit anti-GST pAb (GenScript, A00097), 2 μg/ml of HRP-labeled protein A (Pierce, Cat. No. 32400) for 40 min. Finally the blot was developed with BioFX TMB system (BioFx, Cat. No. TMBM-0100-01). The result is shown in FIG. 11.

### Example II

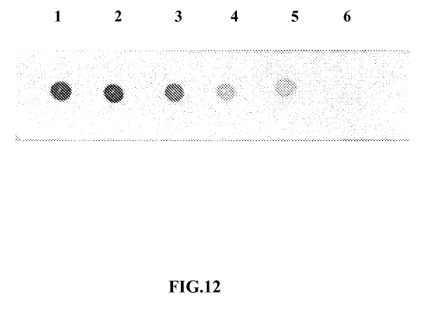

Dot Blot for the detection of IL-8 protein (Genscript, Z00288). 1 μl of 100 ng/μl, 34 ng/μl, 14 ng/μl, 4.5 ng/μl, 1.5 ng/μl and 0.5 ng/μl of IL-8 protein were spotted at 1, 2, 3, 4, 5 and 6 of nitrocellulose membrane, respectively. The membrane was then incubated in GS solution containing 1.5 μg/ml of Anti-Human IL-8 monoclonal Antibody (PIERCE, M801), 0.5 μg/ml of HRP-labeled Anti-Mouse IgG (Fc specific) (Sigma, Cat. No. A0168) for 40 min. Finally the blot was developed with BioFX TMB system (Cat. No. TMBM-0100-01). The results are shown in FIG. 12.

### Example III

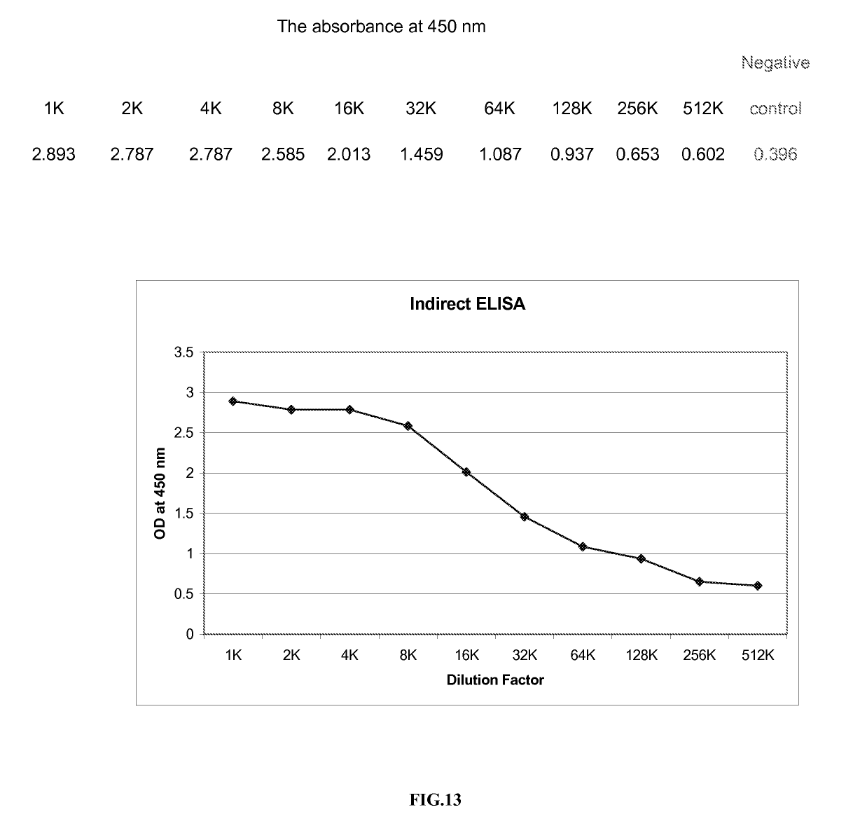

Indirect ELISA for the titration of antibody. Purified GST protein (Genscript, Cat. No. Z02039) was coated on a 96-well plate at 4 μg/ml following standard method. GST Rabbit polyclonal antibody (Genscript, Cat. No. A00097) was diluted and added to the plate wells in GS solution containing 0.1 μg/ml of HRP-labeled Protein A (Pierce, Cat. No. 32400) for 1 hour at 37° C. Finally the plate wells were washed and developed with TMB system (Genscript, Cat. No. T002). The absorbance at 450 nm was measured using a microtiter plate spectrophotometer. The results are shown in FIG. 13.

### Example IV

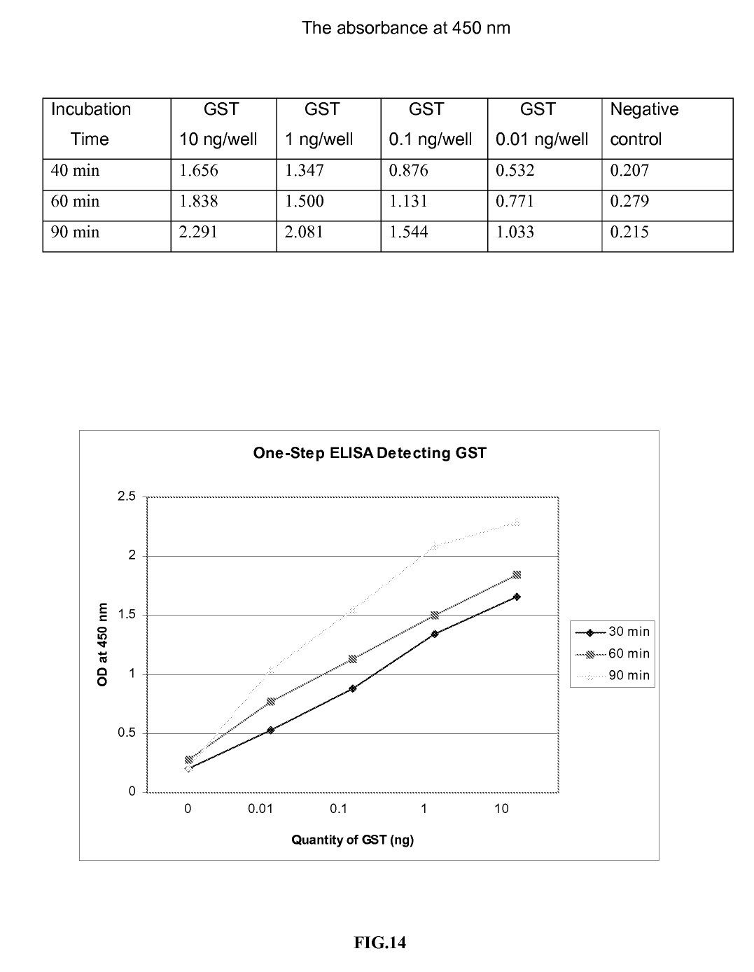

Sandwich ELISA for the detection of antigen. Rabbit polyclonal antibody (Genscript, Cat. No. A00014) was pre-coated on a 96-well plate wells. 0.01 ng, 0.1 ng, 1 ng and 10 ng of GST protein (Genscript, Cat. No. Z02039) were added to and mixed with GS solution containing 1 μg/ml of mouse anti-GST monoclonal antibody (Genscript, Cat. No. A00097) and 0.5 μg/ml of HRP-labeled goat secondary antibody against mouse IgG (Sigma, Cat. No. A0168), respectively. These mixtures were added to the pre-coated plate wells and incubated for 40 min, 60 min and 90 min, respectively. After incubation, plate wells were washed and developed with TMB system (Genscript, Cat. No. T002). The absorbance at 450 nm was measured using a microtiter plate spectrophotometer. The results are shown in FIG. 14.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. A method comprising:
incubating a substrate comprising a target with a solution comprising at least a primary and a secondary detection agent;
washing the substrate; and
detecting the target by measuring the primary and/or secondary detection agent on the substrate.

2. The method of claim 1, wherein the solution comprises Na₂HPO₄, NaH₂PO₄, NaCl, a blocking agent, and the primary and/or secondary detection agent.

3. The method of claim 2, wherein the solution comprises Na₂HPO₄ in the range of about 2 g to 8 g per liter; the amount of NaH₂PO₄ in the range of about 1 gram to 5 gram per liter; the amount of NaCl in the range of about 3 gram to 10 grams per liter; wherein the blocking agent comprises BSA and/or Tween-20; The amount of BSA is in the range of about 5 grams to 30 grams per liter; and the amount of Tween-20 is in the range of about 0.1 grams to 10 grams per liter; the primary and/or secondary detecting agent comprises a reporter-conjugated molecule and wherein the pH is in the range of about 6.0 to about 8.0.

4. The method of claim 1, wherein the substrate is a membrane, a tissue, or a solid phase.

5. The method of claim 1, wherein the substrate comprises a target directly spotted on the substrate.

6. The method of claim 1, wherein the substrate comprises a histology sample.

7. The method of claim 4, wherein the solid phase comprises an ELISA plate.

8. The method of claim 4, wherein the membrane comprises electrophoretically resolved targets.

9. The method of claim 1, wherein the target is selected from the group consisting of a polypeptide, a chemical, an RNA, a DNA, a cell, or a virus particle.

10. The method of claim 9, wherein the polypeptide is an antibody.

11. The method of claim 1, wherein the incubating comprises incubation on a vibration device and/or a shaking or rotational device.

12. The method of claim 1, wherein the target is an HIV antigen.

13. The method of claim 1, wherein the target is an antigen associated with Lyme disease.

14. The method of claim 1, wherein the solution comprises a tertiary detection agent.

15. A method for the detection of an antigen or a detection agent, comprising:
(a) incubating a substrate comprising a target in a solution containing both a primary detection agent and a secondary detection agent;
(b) washing the substrate; and
(c) detecting the substrate from Step (b) with a detection system to observe signals,
wherein the incubation and washing can be conducted on a regular shaker or a vibrator with rotation or shaking.

16. The method of claim 1 or 15, wherein the substrate is first blocked.

17. The method of claim 1 or 15, wherein the substrate comprising the target is first incubated with a blocking agent prior to the incubating with the solution.

18. The method of claim 1 or 2, wherein the primary and/or secondary detection agent is selected from the group consisting of an antigen, an antibody, a chemical compound, a peptide molecule, a protein molecule, an RNA molecule, a DNA molecule, a bacteria cell, a virus particle, or a cell.

19. The method of claim 18, wherein the primary and/or secondary detection agent are labeled.

20. A solution comprising:
about 2 grams to 8 grams per liter of Na₂HPO₄, about 1 gram to 5 grams per liter of NaH₂PO₄, about 3 grams to 10 grams per liter of NaCl, about 5 grams to 30 grams per liter of BSA and/or about 0.1 grams to 10 grams per liter of Tween-20 and a pH of about 6 to 8.

21. The solution of claim 20, further comprising about 0.1 milligrams to 10 milligrams per liter of HRP-labeled secondary antibody.

22. A kit comprising a solution of claim 20 and one or more components for performing a Western Blot, a dot blot, an ELISA, or an immunohistochemistry technique.
